(19) United States
(12) Patent Application Publication
(10) Pub. No.: US 2002/0119163 A1
(43) Pub. Date: Aug. 29, 2002

(54) Methods and compositions for protection against bovine viral deseases

(75) Inventor: Subramaniam Srikumaran, Lincoln, NE (US)

Correspondence Address: SENNIGER POWERS LEAVITT AND ROEDEL, ONE METROPOLITAN SQUARE, 16TH FLOOR, ST LOUIS, MO, 63102, US

(73) Assignee: The Board of Regents of the University of Nebraska

(21) Appl. No.: 10/003,907

(22) Filed: Nov. 2, 2001

Related U.S. Application Data
(60) Provisional application No. 60/245,970, filed on Nov. 3, 2000.

(51) Int. Cl. A61K 39/12

(52) U.S. Cl. 424/186.1

(57) ABSTRACT

The present invention relates to methods and compositions for eliciting an immune response against bovine viral epitopes. The methods comprise combining at least one heat shock protein with at least one bovine viral epitope to form a purified epitope/heat shock protein complex and administration of an immune system stimulating amount of the purified epitope/heat shock protein complex. The compositions comprise, a purified epitope/heat shock protein complex comprising at least one bovine viral epitope complexed with at least one heat shock protein, and a pharmaceutically acceptable carrier, diluent or excipient.

### BACKGROUND

This invention relates to compositions and methods of immunizing animals against bovine viral diseases. In particular, this invention relates to compositions comprising bovine viral epitopes complexed to a heat shock protein and use of said compositions to immunize animals. More particularly, this invention relates to compositions comprising bovine viral epitopes complexed to heat shock glycoprotein 96 and the use of said compositions to immunize animals.

Numerous vaccine preparations utilizing killed (inactivated) or live attenuated viruses are currently available. Inactivated vaccines are prepared by killing the virus, for example, by heat, irradiation or chemical treatment. Many inactivated vaccines have not been satisfactory since they fail to induce humoral immunity of long duration, and are incapable of inducing a satisfactory cell-mediated immune response. Attenuated vaccines are prepared by a large number of passages on homologous or heterologous cells. These passages result in unknown mutations/deletions which reduce the pathogenicity of the virus. Alternatively, attenuated viruses can be produced by genetic engineering. For example, Rijsewijk (U.S. Pat. No. 5,676,951) teaches the use of mutant bovine herpesvirus 1(BHV 1) containing either a naturally occurring or induced deletion in the glycoprotein gE gene. Attenuated virus vaccines give better protection than do killed virus vaccines, however, attenuated vaccines set up latent infections in vaccinated animals which may lead to disease outbreak if the virus reactivates and mutates back to a virulent form.

To overcome the shortcomings of killed or attenuated virus vaccines, attempts have been made to provide vaccines based on viral proteins or nucleotide sequences encoding viral proteins. Israel et al. (Vaccine, 6:349-356, 1988) discloses the use of a BHV 1 glycoprotein vaccine. Although administration of this vaccine resulted in high antibody titers, it did not confer resistance to an intra nasal challenge of BHV 1.

Because of the ability of many viruses to spread from cell to cell, cell mediated immunity, specifically that provided by virus-specific cytotoxic T lymphocytes (CTLs), is equally if not more important than neutralizing antibodies in protecting the animal from infection. To be recognized by CTLs, an infected cell must present the viral peptides in association with the Major Histocompatibility Complex (MHC) class I molecules on its surface. These CTL peptide epitopes are generated by proteosomal processing of viral proteins, and the epitopes are translocated from the cytosol into the lumen of the endoplasmic reticulum by specialized transporters called “transporters associated with antigen processing” (TAP). The 8 to 10-mer CTL peptide epitopes associate and form a stable complex with the class I heavy chain and the β₂-microglobulin in the endoplasmic reticulum. The family of peptides bound by a particular class I molecule is characterized by the presence of a restricted number of “anchor” amino acid residues, at particular positions in the peptide. The “anchor” residues and their position in the peptides bound by a particular class I allelic product constitute the “allele-specific peptide motif” (ASPM). Thus, peptides containing ASPMs are especially effective in eliciting cell mediated immune responses. The extreme polymorphism in the MHC, however, can result in a large number of possible alleles, and hence ASPMs. Thus, limiting their potential therapeutic value.

The discovery of MHC supertypes in humans presents a possible solution to the problem presented by MHC polymorphism. Supertypes are a group of class I alleles which share the same or similar ASPMs. The term supermotif is used to refer to such motifs which bind to a large number of different class I alleles (Sette and Sidney, Curr. Opin. Immunol., 10:478-482, 1998). By using peptides containing supermotifs, it is possible to bind epitopes to a wide variety of MHC class I molecules, and hence elicit a CTL response in a large percentage of individuals in a population.

It is now known that there are other transporters involved in epitope presentation. These transporters are members of the class of proteins known as heat shock proteins (HSPs) (See, Schild et al., Curr. Opin. Immunol., 11:109-113, 1999). Heat shock proteins are a group of proteins whose presence was originally associated with cell stress, particularly increased temperature. Three major families of HSPs hsp60, hsp70 and hsp90 have been identified on the basis of their molecular weights (Welch, Scientific American, 56-64, May 1993). Many members of these families have been found to be induced in response to stressful stimuli in addition to heat stress such as nutrient deprivation, metabolic disruption and intracellular pathogens (Welch, Scientific American, 56-64, May 1993; Craig, Science, 260:1902-1903, 1993; Gething, et al., Nature, 355:33-45, 1992; Young, Ann. Rev. Immunol., 8:401-420, 1990; Lindquist et al., Ann. Rev. Genet., 22:631-677, 1988).

Heat shock proteins are highly conserved among species. For example, hsp70 shows 74% nucleotide sequence homology between yeast and Drosophila, and 85% sequence homology between Drosophila and mice (Moran et al., Can. J Biochem. Cell Biol., 61:488-499, 1983). The amino acid sequence of human hsp70 is 40% identical to E. coli hsp70, dnaK, and 73% identical to Drosophila hsp70 (Hunt and Morimoto, Proc. Natl. Acad. Sci. USA, 82:6455-6459, 1985). Thus, the present invention contemplates the use of HSPs not only within species (homologous HSP), but also across species (heterologous HSPs).

The evidence that HSPs are involved in immune system function came from the observation that HSPs isolated from cancer cells or virus infected cells induced protective immunity or cytotoxic T lymphocytes (CTL) to the cognate tumor or viral antigen. In contrast, HSPs isolated from non-cancerous or uninfected cells elicited no immune response. This, combined with the finding that the HSPs do not show tumor-associated DNA polymorphism, suggested that HSPs were not immunogenic themselves, but served as chaperones for peptides formed during antigen processing (Suto and Srivastava, Science, 269:1585-1588, 1995). Members of all three HSP families, hsp60, hsp70 and hsp90, have been shown to play a role in stimulation of cell mediated immunity (Könen-Waisman et al., J. Infect. Dis., 179:403-413, 1999; Schild et al., Curr. Opin. Immunol., 11:109-113, 1999; Blachere et al., J. Exp. Med., 186:1315-1322, 1997; Heike et al., J. Leukoc. Biol., 60:153-158, 1996). It has been suggested that HSPs complexed with antigenic peptides are released from virus infected or cancerous cells by lysis of the cells during infection or by the action of antibodies or nonspecific effectors. The HSP/antigenic peptide complexes are then taken up by macrophages or other specialized antigen-presenting cells, possibly by a receptor mediated mechanism. The complex is then routed to the endogenous presentation pathway in the antigen presenting cell and is displayed in the context of that cell's MHC class I, where it is recognized by CTLs (Srivastava et al., Immunogenetics, 39:93-98, 1994; Suto and Srivastava, Science, 269:1585-1588, 1995). More recently, however, this suggestion has been questioned (Schild et al., Curr. Opin. Immunol., 11:109-113, 1999) based on antisense experiments in which inhibition of gp96 expression failed to influence the ability of cells to present peptides to CTLs (Lammert et al., Eur. J. Immnol. 26:875-879, 1996). In addition, it has been found that the HSP gp96 cannot bind peptides with charged amino acids at P2 and P9 (Spee and Neefjes, Eur. J. Immunol. 27:2441-2449, 1997) and that gp96 has a hydrophobic peptide binding domain (Wearsch et al., Biochemistry, 37:5709-5719 (1998) thus limiting its peptide binding.

Glycoprotein 96 (gp96) is a member of the HSP 90 family which is found in the endoplasmic reticulum. Glycoprotein 96 preparations isolated from cells expressing a transfected cytosolic protein have been found to elicit specific CTLs against that antigen (Arnold et al., J. Exp. Med., 182:885-889, 1995). In virus infected cells, gp96 preparations isolated from cells infected with vesicular stomatitis virus (VSV) were found to contain VSV derived peptides (Nieland et al., Proc. Natl. Acad. Sci. USA, 93:6135-6139, 1996). For vaccine production, it has been found that gp96-peptide complexes can be generated in vitro and that these complexes elicit immunity by a mechanism apparently identical to that seen with in vivo generated complexes.

Interest in gp96 as an aid to inducing an immune response against an antigen comes from studies which found that immunization of mice with gp96 isolated from tumor cells provided protection against a subsequent challenge with the tumor cells from which the gp96 was isolated (Srivastava et al., Proc. Natl. Acad. Sci. USA, 83:3407-3411, 1986; Srivastava et al., Adv. Cancer Res., 62:153-177, 1993). Further studies demonstrated that immunization with gp96 molecules from autologous tumor cells elicited CD4⁺ and CD8⁺ T cell responses (CD=Cluster of Differentiation) against the primary tumor and its metastasises (Tamura et al., Science, 278:117-120, 1997. Srivastava teaches the use of antigens bound to gp96 for the immunotherapeutic treatment of cancer (U.S. Pat. Nos. 5,830,464, 5,837,251, 5,935,576, 5,948,646 and PCT publications WO 97/10001, WO 98/34641). The same publications disclose the use of gp96/antigen complexes to treat or prevent various infectious diseases in humans. Srivastava, however, does not teach or suggest the use of gp96 as an adjuvant to induce CTLs against bovine viruses nor does he teach the use of viral sequences that contain ASPMs. In addition, Srivastava does not show stimulation of a humoral as well as a cell-mediated response.

### SUMMARY

To be effective, it is widely accepted that any vaccine preparation must also stimulate a cell mediated immune response, and in particular, the activation of cytotoxic T lymphocytes. Presently, attenuated virus vaccines are used to stimulate cell mediated immunity. As discussed previously, attenuated viral vaccines are not without problems. There is a need, therefore, for a composition that is capable of eliciting a cell mediated response against bovine viruses, but does not involve the use of a live virus. The present invention meets that need.

The present invention insures a T cell response by utilizing the heat shock protein gp96 as an adjuvant. The gp96 protein is involved in the association of peptides with the MHC type I presentation pathway. Presentation of the antigen by the MHC class I complex is thought to be critical for eliciting a cell-mediated immune response. The fact that gp96 transfers peptides to the class I antigen presentation pathway ensures that the peptide epitopes complexed to it will be directed to the class I antigen presentation pathway. Also, because only peptides are used, there is no problem with virus reactivation and shedding, down regulation of the MHC class I surface molecules, and apoptosis of CD4⁺ T cells as is seen with modified live virus preparations that are currently used to stimulate cell mediated immunity against BHV 1.

In addition, the method of obtaining epitope/heat shock protein complex from a transfected cell expressing a BHV 1 protein alleviates the need for prior identification of specific CTL epitopes. In this method, the epitope/heat shock protein complex contains not only the peptides presented by the class I molecules of the transfectant, but also the peptides presented by other class I alleles as well. The epitope/heat shock protein complex, therefore, can be used to immunize animals with a different MHC background.

Accordingly, among the aspects of the present invention is to provide a method for eliciting an immune response in an animal to a bovine virus comprising, combining a bovine viral epitope and a heat shock protein to form a purified epitope/heat shock protein complex and administering an immune system stimulating amount of said purified epitope/heat shock protein complex to an animal.

Another aspect of the invention, is a composition comprising at least one bovine viral epitope complexed to a heat shock protein to form a purified epitope/heat shock protein complex, and a pharmaceutically acceptable carrier, diluent or excipient.

In another aspect of the invention, the bovine viral epitope contains an allele specific peptide motif.

In still another aspect of the invention, the bovine viral epitope contains a supermotif.

In yet another aspect of the invention, the allele specific peptide motif is selected from the group consisting of BoLA-A11, BoLA-A20, BoLA-HD1, BoLA-HD6, and BoLA-HD7.

Yet another aspect of the invention provides a method for producing a bovine viral CTL epitope/heat shock protein complex by transfecting a cell with a nucleotide sequence encoding a bovine viral protein, inducing expression of the bovine viral epitope under conditions which also induce expression of the heat shock protein and isolating the epitope/heat shock protein complex from the cells.

Still another aspect of the invention is a method for producing a bovine viral epitope/heat shock protein complex comprising combining an isolated heat shock protein with at least one bovine viral epitope and isolating the complex from the uncomplexed epitope and heat shock protein.

### Definitions

HSP=heat shock protein

ASPM=allele specific peptide motif

BSA=bovine serum albumin

CTL=cytotoxic T lymphocyte

gp96=heat shock protein glycoprotein 96

BHV 1=bovine herpesvirus 1

β-gal=β-galactosidase

Triton X-100=t-Octylphenoxypolyethoxyethanol

Tween-20=polyoxyethylenesorbitan monolaurate

CD=Cluster of Differentiation

moi=multiplicity of infection

pfu=plaque-forming unit

cpm=counts per minute

FBS=fetal bovine serum

PBMC=peripheral blood mononuclear cell

As used herein, “epitope” means a single antigenic determinant of an antigenic molecule that stimulates a specific immune response and against which that response is directed. As used herein, the term includes not only the determinant, but also the molecule or fragment of the molecule which contains the determinant.

As used herein the term “purified epitope/heat shock protein complex” means that the complex is separated from the majority of cell proteins normally associated with it or that the complex is synthesized in purified form. Purity may be assayed by standard methods, and will ordinarily be at least about 50% pure, generally at least about 60% pure, more generally at least about 70% pure, often at least about 75% pure, more often at least about 80% pure, typically at least about 85% pure, more typically at least about 90% pure, preferably at least about 95% pure, more preferably at least about 98% pure, and most preferably, at least 99% pure. The analysis may be weight or molar percentages, evaluated, e.g., by gel staining, spectrophotometry, or terminus labeling.

As used herein, “naive” refers to an animal or cell that has not been previously exposed to the antigen in question.

As used herein, the term “epitope/heat shock protein complex” refers to a complex containing at least one epitope and at least one heat shock protein.

### DETAILED DESCRIPTION

All publications, patents, patent applications and other references cited in this application are herein incorporated by reference in their entirety as if each individual publication, patent, patent application or other reference were specifically and individually indicated to be incorporated by reference.

Applicants have invented methods and compositions for eliciting an immune response in an animal to at least one epitope of a bovine virus. In particular, at least one epitope of a bovine virus is complexed to a heat shock protein which is then administered to an animal in an amount that stimulates a measurable immune response (immune system stimulating amount). Methods for determining stimulation of the immune system are well known to those of ordinary skill in the art. Methods include, but are not limited to, the determination of circulating antibodies and/or the presence of specific cytotoxic T lymphocytes. Stimulation of CTLs is thought to be critical to providing immunity to viruses.

The complexes of the present invention can be comprised of any combination of heat shock proteins and epitopes of bovine viruses in any manner of binding association. In one embodiment, an epitope is non-covalently bound to a heat shock protein. In another embodiment, the epitope contains an allele-specific peptide motif (ASPM). In yet another embodiment, the epitope contains a supermotif. Suitable heat shock proteins include members of the heat shock protein 60, heat shock protein 70, and heat shock protein 90 families. The heat shock protein can be obtained from the species to which it is to be administered (homologous heat shock protein) or it can be from a different species (heterologous heat shock protein). In one preferred embodiment, the heat shock protein used is glycoprotein 96 (gp96). Glycoprotein 96 is a glycosylated member of the heat shock protein 90 family.

Any protein or peptide derived from the bovine virus of interest can be used in complex with a heat shock protein. Examples of viruses from which proteins can be obtained include, but are not limited to, bovine viral diarrhea virus, bovine respiratory syncytial virus, parainfluenza virus III, bovine corona virus, and bovine rota virus. Particularly useful are proteins and peptides containing allele-specific peptide motifs (ASPM). Preferred ASPMs include bovine lymphocyte antigens (BoLA)-A11 (Hegde et al., Immunogenetics, 42:302-303, 1995), BoLA-A20 (Bamford et al., Immunol Lett., 45:129, 1995) and BoLA-HD1, -HD6 and -HD7 (Gaddum et al., Immunogenetics, 43:238, 1996). Because the peptides usually presented by the MHC class I complex are 8 to 10-mers, the epitopes used can comprise fragments of bovine viral proteins. In one embodiment, epitopes comprise peptides of between 5 and 25 amino acids in length. In another embodiment, epitopes comprise peptides of between 5 and 15 amino acids in length. In yet another embodiment, epitopes comprise peptides of between 8 and 10 amino acids in length.

The preparation can be administered to any animal which can become infected with a bovine virus. In one embodiment, the animal is a ruminant animal, more preferably a Bovidae and more preferably still a member of the genus Bos.

The animal can be administered complexes comprising a single epitope complexed to HSPs or can be administered complexes comprising multiple epitopes complexed to HSPs. The epitope/HSP complexes can be administered in a single dose or the initial dose can be followed by one or more booster doses. If more than one epitope is used, then all epitopes can be given in each administration or, alternatively, different epitope/HSP complexes can be given at each administration.

The complexes of the present invention can be administered by a variety of routes and methods. Suitable routes and methods of administration include orally, parenterally, by inhalation spray, rectally, intradermally, transdermally, or topically in dosage unit formulations containing conventional nontoxic pharmaceutically acceptable carriers, adjuvants, and vehicles as desired. The term parenteral as used herein includes subcutaneous, intravenous, intramuscular, or intrasternal injection, or infusion techniques. In one embodiment, the complexes are administered by injection and more particularly by intramuscular injection. In another embodiment, the complexes are administered by an intra nasal inhalation spray. In yet another embodiment, the complexes can be administered by multiple routes, as for example is taught in U.S. Pat. No 5,462,734. Methods for the formulation of drugs is well known in the art and is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful.

Suppositories for rectal administration of the compounds discussed herein can be prepared by mixing the active agent with a suitable non-irritating excipient such as cocoa butter, synthetic mono-, di-, or triglycerides, fatty acids, or polyethylene glycols which are solid at ordinary temperatures, but liquid at the rectal temperature, and which will therefore melt in the rectum and release the complex.

Solid dosage forms for oral administration may include capsules, tablets, pills, powders, and granules. In such solid dosage forms, the compounds of this invention are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, or magnesium or calcium carbonate or bicarbonate. Tablets and pills can additionally be prepared with enteric coatings.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. The compounds can be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The amount of epitope/HSP complex that can be combined with the carrier materials to produce a single dosage form will vary depending upon the patient or animal and the particular mode of administration.

Any suitable method known in the art can be used to obtain the bovine viral epitopes or HSPs used in the present invention. In general, three methods can be used. In one method, the bovine viral epitopes and/or HSPs can be isolated from cells which naturally produce the epitopes or HSPs. For example, suitable susceptible host cells can be infected with the virus of interest and the cells grown in culture. In one method, virus is then isolated from the host cells and the epitopes isolated from the virus. The isolated virus is treated with a detergent to release the glycoproteins located within the lipid envelop of the virion. Alternatively, glycoprotein epitopes present on the surface of infected cells can be obtained by detergent-solubilized lysates of infected cells rather than from whole virions. The proteins are then separated from the detergent and other debris and the individual protein epitopes isolated by methods well known to those of ordinary skill in the art.

Glycoprotein 96 can be purified from any cell that naturally expresses the protein. The gp96 can be isolated from tissues collected in vivo or can be from cells grown in vitro. If obtained from in vitro cell culture, conditions can be manipulated, for example increased temperature, to induce increased production of gp96. Various methods for obtaining proteins from cells and tissue are known to those of skill in the art. These include precipitation by, for example, ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, high performance liquid chromatography (HPLC), electrophoresis under native or denaturing conditions, isoelectric focusing, and immunoprecipitation.

In one embodiment, gp96 is isolated from liver cells using the method described by Srivastava, Methods, 12:165-171, 1997. Briefly, liver cells are homogenized in a solution of 30 mM sodium bicarbonate, pH 7, and 1 mM phenylmethane-sulphonyl fluoride (PMSF) using a mechanical homogenizer such as a Polytron. The lysate is centrifuged at 2000 g and 4° C. to remove cellular debris. The supernatant is re-centrifuged at 100,000 g for 90 minutes at 4° C. Gp96 can be isolated either from the pellet or supernatant from this centrifugation. Purification of gp96 from the supernatant is accomplished by bringing the supernatant to 50% ammonium sulfate and stirring for 2 to 12 hours at 4° C. followed by centrifugation at 6000 rpm in a SS34 rotor. The supernatant from this centrifugation is brought to 70% ammonium sulfate and centrifuged as for the 50% cut. The resulting pellet is washed in PBS containing 70% ammonium sulfate and then dissolved in 10 volumes of PBS containing 2 mM each Ca²⁺ and Mg²⁺ (Ca/Mg PBS). Any undissolved material is removed by centrifugation. The dissolved solution is then added to a concanavalin A chromatography column and the bound proteins eluted with 10% α-D-methyl mannoside dissolved in Ca/Mg PBS. One third of the column volume is applied to the column after which the column is sealed and incubated at 37° C. for 30 minutes. Following this incubation, five column volumes of eluant are applied and the fractions collected. Protein containing fractions are then applied to a DEAE ion exchange column and the proteins eluted with five volumes of 700 mM NaCl, 5 mM sodium phosphate, pH 7. For purification of gp96 from the 100,000 g pellet, the pellet is suspended in 5 volumes of PBS containing 0.1% octy glucopyranoside and placed on ice for 1 hour. The suspension is then centrifuged at 20,000 g for 30 minutes at 4° C. and the detergent removed by dialysis against PBS or other suitable method. The resulting solution is centrifuged at 100,000 g for 90 minutes and calcium and magnesium are added to the supernatant to a final concentration of 2 mM each. Further purification is carried out as previously described.

In a second method, epitopes and/or HSPs can be made by recombinant DNA technology. Once the nucleotide sequence encoding the viral epitope or HSP of interest is known, it can be placed into an expression vector and used to transfect a suitable host cell by methods commonly known to those of ordinary skill in the art. Sambrook et al., Molecular Cloning, A Laboratory Manual, 2_{nd} Ed., Cold Spring Harbor Press, (1989) and Ausubel et al., Short Protocols in Molecular Biology, 2_{nd} Ed., John Wiley & Sons (1992). Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used.

The nucleotide sequence of interest may be inserted into the vector by a variety of methods. In the most common method, the sequence is inserted into an appropriate restriction endonuclease site(s) using procedures commonly known to those skilled in the art and detailed in, for example, Sambrook et al., Molecular Cloning, A Laboratory Manual, 2_{nd} Ed., Cold Spring Harbor Press, (1989) and Ausubel et al., Short Protocols in Molecular Biology, 2_{nd} Ed., John Wiley & Sons (1992).

In an expression vector, the sequence of interest is operably linked to a suitable expression control sequence or promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control. Promoters are generally classified as either inducible or constitutive. Inducible promoters are promoters that initiate increased levels of transcription from DNA under their control in response to some change in the environment, e.g. the presence or absence of a nutrient or a change in temperature. Constitutive promoters, in contrast, maintain a relatively constant level of transcription.

A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2_{nd} Ed., Cold Spring Harbor Press, (1989) and Ausubel et al., Short Protocols in Molecular Biology, 2_{nd} Ed., John Wiley & Sons (1992).

Common promoters used in expression vectors include, but are not limited to, LTR CMV or SV40 promoter, the E. coli lac or trp promoters, and the phage lambda PL promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

Expression vectors can, and usually do, contain a selection gene or selection marker. Typically, this gene encodes a protein necessary for the survival or growth of the host cell transformed with the vector. Examples of suitable markers include dihydrofolate reductase (DHFR) or neomycin resistance for eukaryotic cells, and tetracycline or ampicillin resistance for E. coli.

In addition, expression vectors can also contain marker sequences operatively linked to a nucleotide sequence for a protein that encode an additional protein used as a marker. The result is a hybrid or fusion protein comprising two linked and different proteins. The marker protein can provide, for example, an immunological or enzymatic marker for the recombinant protein produced by the expression vector. Numerous suitable vectors are commercially available and are known to those of ordinary skill in the art.

Once an expression vector has been constructed it is placed into a suitable host cell. The host cell will vary with the vector used, but in general can be a higher eukaryotic cell, such as a mammalian cell, or a lower eukaryotic cell such as a yeast cell, or the host can be a prokaryotic cell such as a bacterial cell. Introduction of the construct into the host cell can be accomplished by a variety of methods including calcium phosphate transfection, DEAE-dextran mediated transfection, Polybrene, protoplast fusion, liposomes, direct microinjection into the nuclei, scrape loading, and electroporation.

Once transfected host cells are selected based on expression of the sequences encoded by the vector. Selected host cells are then grown in culture and induced to produce the protein of interest. If the host cell secretes the protein into the culture medium, then the protein can be purified from the medium. If the protein is not secreted, the host cells can be harvested, lysed and the protein collected from the lysate. Proteins can be purified from the cell medium or lysate by any suitable method, such as those previously discussed.

The vector can be constructed so as to produce a single protein of interest or several proteins. For example, a fusion protein of several bovine viral epitopes can be constructed. The resulting protein can be used as a fusion protein or be constructed such that the epitopes can be separated, for example, by enzymatic cleavage.

A third method for the production of BHV 1 epitopes or HSP is by chemical synthesis. Any method of peptide synthesis can be used to practice the present invention. Chemical synthesis of peptides is well known to those of ordinary skill in the art (Bodanszky, Principles of Peptide Synthesis, Springer-Verlag, 1993; Atherton and Sheppard, Solid Phase Peptide Synthesis: A Practical Approach, IRL Press, 1989; Bodanszky and Bodanszky, The Practice of peptide Synthesis, Springer-Verlang, 1984). Modern solid-phase peptide synthesis involves the creation of a linear peptide chain by the successive addition of amino acids to a growing peptide whose C terminus is covalently linked to a solid support or resin. Solid-phase peptide synthesis entails three repeated reactions, deprotection, activation, and coupling. In order to prevent unwanted reactions at their alpha and side-chain functionalities, amino acids used in peptide synthesis are derivatized or “protected.” Commonly used amino protecting groups include the t-butoxycarbonyl group (Boc), 9-fluorenylmethyloxycarbonyl (Fmoc), 2-(4-biphenylyl)propyl(2)oxycarbonyl (Bpoc), 2-ntiro-phenylsulfenyl (Nps), and dithia-succionyl (Dts). During the deprotection step, the protecting group is removed to make the alpha-amino group on the end of the growing peptide chain available. Activation converts the next amino acid to be added into an active ester. During coupling, an amide bond is formed between the active ester and the deprotected alpha-amino group. The process is repeated until the desired peptide is formed. When synthesis is completed, the side-chain protecting groups are removed and the peptide cleaved from the solid phase. In one preferred embodiment, bovine viral epitopes are made by solid phase peptide synthesis using standard Fmoc chemistry.

Viral epitopes useful in the practice of the present invention preferably are capable of binding to a heat shock protein. Several methods are available to determine binding. One method for determination of binding is that used by Blachere et al., J. Exp. Med., 186:1315-1322, 1997. In general, the epitope to be tested is coupled to a detection moiety. Numerous detection moieties will be readily apparent to those of ordinary skill in the art and include, without limitation, radioactive labels, such as radionuclides, fluorophores or fluorochromes, peptides, enzymes, antigens, antibodies, vitamins or steroids. In one embodiment, the detection moiety is a radionuclide, preferably ¹²⁵I. The epitope to be tested is then incubated with a heat shock protein under conditions which promote binding of the epitope to the heat shock protein. In one embodiment, binding is conducted at room temperature for 30 minutes in a binding buffer comprising 20 mM HEPES, pH 7, 20 mM NaCl and 2 mM MgCl₂. After the binding reaction, bound and unbound labeled epitopes are separated by any suitable means, for example, ultrafiltration or column chromatography. The amounts of bound and unbound labeled epitope are then determined, such as in one embodiment, by quantitative autoradiography to determine the amount of binding. It will be apparent to those skilled in the art, that screening for binding can also be accomplished by using a labeled heat shock protein rather than a labeled epitope. Also, it may be possible to determine binding without the use of a label coupled to one of the proteins by conducting a binding reaction and then separating the reaction products, by for example SDS-PAGE electrophoresis and staining. The binding of the epitope to the heat shock protein will result in a band of increased molecular weight.

In the present invention, an immune response to a viral epitope is achieved by administration of the epitope in combination with a heat shock protein. In one embodiment, the heat shock protein is gp96. The heat shock protein can be bound to the epitope by any method known in the art. The binding reaction can be conducted in vitro by the method described by Blachere et al., J. Exp. Med., 186:1315-1322, 1997, and discussed above. When the HSP/epitope complex is formed in vitro, the ratio of epitope to HSP used to form the complex can vary over a wide range. In one embodiment, the ratio of epitope to HSP is one part epitope to 100 to 100,000 parts HSP. In another embodiment, the ratio is one part epitope to 1,000 to 50,000 parts HSP. In yet another embodiment, the ratio is one part epitope to 10,000 to 25,000 parts HSP and in still another embodiment, the ratio is one part epitope to 12,500 parts HSP.

Alternatively the epitope-HSP complex can be produced in cells. In this embodiment, a host cell that expresses a heat shock protein is transfected with a vector containing a nucleotide sequence encoding the viral protein or epitope of interest. Methods for producing expression vectors and for transforming host cells have been discussed previously. Transformed cells are induced to express both the endogenous heat shock protein and the exogenous viral epitope. The cells are then harvested, lysed and the epitope-HSP complexes isolated using standard protein purification techniques such as those previously discussed.

The ability of epitope/HSP complexes to elicit cytotoxic T lymphocytes can be determined, for example, by the ⁵¹Cr release assay. Methods for conducting this assay are known to those of ordinary skill in the art and can be found, for example, in Zatechka et al., Vaccine, 17:686-694, 1999 and Stikovsky and Henkart, eds., Cytotoxic Cells, Birkauser, 1993. To generate cytotoxic T lymphocytes, animals are twice administered at 1 to 3 week intervals, the candidate epitope/HSP complex, the HSP protein alone or the epitope alone. Five to ten days after the second administration, the animals are euthanized and the spleens collected. Lymphocytes are isolated by any suitable method, for example, by use of a Percoll gradient. Lymphocytes are restimulated in vitro with peptide epitope pulsed, naive, syngeneic, stimulator lymphocytes. Stimulator lymphocytes can be prepared, for example, by pulsing with from 10 to 200 μg/ml of epitope for 1 hour at 37° C. in any suitable culture medium, for example, RPMI-1640 supplemented with 10% fetal bovine serum. In one preferred embodiment, stimulator lymphocytes are pulsed with 100 μg/ml of epitope. The pulsed stimulator lymphocytes are irradiated with 2000 Rad. Any suitable source of radiation can be used. In one embodiment, a ⁶⁰CO irradiator is used. Responders and stimulators are co-cultured at 2×10⁷ cells of each type at 37° C. in an appropriate culture medium.

Target cells are pulsed with candidate epitopes, either alone or in combination, as described above for the generation of cytotoxic T lymphocytes. Pulsed and non-pulsed target cells are labeled with 100 μCi of ⁵¹Cr per 1×10⁶ cells for one hour at 37° C. Labeled cells are then washed and incubated at different effector:target cell ratios, usually ranging from 1:1 to 100:1 for 4 to 6 hours in a suitable culture medium, for example RPMI-1640 containing 5% fetal bovine serum. Each assay point is preformed in duplicate or triplicate, and appropriate controls to measure spontaneous ⁵¹Cr release (no lymphocytes (effectors) added) and 100% release (lysed target cells). Lysis of target cells can be accomplished, for example, by use of a detergent. In one embodiment, target cells are lysed by treatment with 0.5% Triton-X100. Target lysis is determined by measuring the amount of ⁵¹Cr in a sample of the culture medium using a gamma counter. The percent specific lysis is calculated as follows:

$$\% specific lysis = \frac{\begin{matrix}{sample }^{51} Cr release - \\ {spontaneous }^{51} Cr release\end{matrix}}{\begin{matrix}{maximum}^{ 51} Cr release - \\ {spontaneous}^{ 51} Cr release\end{matrix}} \times 100$$

### EXAMPLES

The following examples are intended to provide illustrations of the application of the present invention. The following examples are not intended to completely define or otherwise limit the scope of the invention.

### Example 1

### Generation of gp96/Epitope Complexes

Epitopes

Epitope peptides are selected to contain ASPMs or supermotifs for the species in which they are to be injected. Epitope peptides are synthesized using standard F-moc solid phase chemistry and purified. If desired, the sequence of the epitope peptides can be confirmed by fast atom bombardment-mass spectrum analysis. Epitope peptides are dissolved in distilled water at 2 mg/ml and stored at −70° C. until used.

Gp96

Heat shock protein gp96 is obtained from liver cells. The procedure used is that described previously. Briefly, liver tissue is homogenized in a solution of 30 mM sodium bicarbonate, pH 7, and 1 mM phenylmethane-sulphonyl fluoride (PMSF), centrifuged and the protein in the supernatant precipitated with 50% and 70% ammonium sulfide. The precipitate from the 70% ammonium sulfate cut is washed, resuspended and further purified by concanavalin A and DEAE chromatography. The gp96 is stored in phosphate buffered, pH 7, 700 mM NaCl at −80° C.

Gp96/Epitope Binding

The peptide binding is carried out as previously described. Two ng of each epitope peptide is mixed with 25 μg of gp96 and incubated at 60° C. for 10 minutes in peptide binding buffer (20 mM HEPES, pH 7, 20 mM NaCl, 2 mM MgCl₂) to dissociate endogenous liver peptides bound to the gp96 molecule. The temperature is then lowered to room temperature followed by an additional incubation for 30 minutes to bind the epitope peptides to the gp96. A control complex is made in which bovine serum albumin (BSA) replaces the gp96 protein.

Immunization and Generation of Cytotoxic T Lymphocytes

Animals are immunized subcutaneously with gp96/epitope complex (25 μg gp96/2 ng epitope), gp96 alone (25 μg), epitope peptide alone (2 ng) in 200 μl, or BSA/epitope complex (25 μg BSA/2 ng epitope). Animals are given two injections spaced one week apart. One week following the last injection, the spleen is collected and lymphocytes isolated.

Lymphocytes are restimulated in vitro with the epitope peptide pulsed (100 μg/ml) naive, syngeneic stimulator cells. Stimulator cells are prepared by pulsing with 100 μg/ml of the epitope peptides in T-cell culture medium (RPMI-1640, 2 mM L-glutamine, 0.1 mM MEM nonessential amino acids, 2.85 g/l NaHCO₃, 1 mM sodium pyruvate, 10 mM HEPES, 50 μM 2-mercaptoethanol, 10% FBS) for 1 hour at 37° C. in a 5% CO₂ atmosphere. The pulsed stimulator cells are then irradiated (2000 Rad) with a ⁶⁰ Co irradiator. Responder and stimulator cells are co-cultured at 2×10⁷ cells of each cell type in 25 cm² culture flasks in T-cell culture medium at 37° C. in a atmosphere of 5% CO₂.

⁵¹Cr Release Cytotoxicity Assay

Syngeneic target cells are pulsed with either the epitope peptides (100 μg/ml) for 1 hour at 37° C. in 5% CO₂. Pulsed and non-pulsed target cells are labeled with 100 μCi of Na⁵¹CrO₄ per 1×10⁶ cells for 1 hour. Labeled cells are washed with RPMI-1640 containing 5% FBS and incubated at effector:target ratios of 10:1, 20:1, 40:1 and 80:1 for 5 hours. Target cell lysis is measured by counting 100 μl of culture medium in an automated gamma counter. The percent specific lysis is calculated as previously described. Spontaneous release is calculated using culture medium from wells containing target cells alone. Maximum release is calculated using culture medium from wells in which the cells add been lysed by 0.5% Triton-X100.

### Example 2

### Cytotoxic T Lymphocyte Recognition in Infected Target Cells

The cytotoxicity assays of Example 1 is conducted with targets pulsed with epitope peptides. To confirm that the results are applicable to virus infected cells, the cytotoxicity assay of Example 1 is repeated with either virus infected or mock infected target cells. For mock infection, culture medium without virus is used. The assay is performed at an effector to target ratio of 20:1.

### Example 3

### Induction of Cytotoxic T-lymphocytes Using gp96/epitope Complexes Generated In Cells

Production of Bovine Viral Epitope Expressing Cells

BC10ME cells (mouse embryo fibroblasts, H-2^{d}) are transfected with a Moloney murine retrovirus vector containing a gene encoding a viral protein or the β-galactosidase gene to yield BC-Pr or BC-βgal cells. These cells constitutively express either a bovine viral epitope protein or β-galactosidase along with gp96. The transfected cells are maintained in Dulbecco's Modified Eagle's Medium (DMEM) containing 4.5 mg/ml glucose, 10% fetal bovine serum, 1 mM sodium pyruvate, 2 mM glutamate, 100 U/ml each of penicillin and streptomycin at 37° C. in a 5% CO₂ atmosphere.

Isolation of Viral Epitope/gp96 Complexes

Cells are lysed in four volumes of a solution of 30 mM sodium bicarbonate, pH 7, and 1 mM phenylmethane-sulphonyl fluoride (PMSF) using a mechanical homogenizer. Complexes are isolated by ammonium sulfate precipitation, concanavalin A chromatography, and DEAE chromatography as described in Example 1.

Immunization and Generation of Cytotoxic T Lymphocytes

Female BALB/c (ByJ) mice (H-2^{d}), 8-12 weeks of age, are given two subcutaneous injections of 25 μg of gp96 isolated from BC-Pr or BC-βgal cells. The injections are given one week apart. Eight days after the second injection, the mice are euthanized, and spleenocytes collected and restimulated in vitro with BC-Pr cells using the methods described in Example 1.

⁵¹Cr Release Cytotoxicity Assay

Five to seven days after restimulation, ⁵¹Cr cytotoxicity assays are conducted according to the method described in Example 1 using BC-Pr and BC-βgal cells as the targets.

### Example 4

### Induction of a Humoral Immune Response by gp96/Epitope Complexes

Immunization

Animals are immunized with two subcutaneous injections of 25 μg of BHV 1 gD/gp96 complex isolated from BC-Pr cells as described in Example 3. Eight days after that last immunization, blood samples are collected, the plasma separated and presence of viral epitope gD antibodies detected by a radioimmunoassay (RIA).

### Example 5

### In vitro Stimulation of Naive Bovine Splenocytes with gp96-Epitope Complexes

In vitro Stimulation

Peripheral blood mononuclear cells (PBMCs) are isolated from naive calves. PBMCs are stimulated in vitro with gp96 derived from BC-Pr cells. Production of BC-Pr cells and isolation of gp96 from BC-Pr cells is described in Example 3. Cells are stimulated by adding 25 μg of gp96 from BC-Pr cells per 1×10⁸ bovine PBMCs in a volume of 1 ml of T cell culture medium (RPMI-1640, 10% FBS) followed by a 1 hour incubation at 37° C. Following stimulation, cells are resuspended in 50 ml of T-cell culture medium, divided among five, 25 cm² tissue culture flasks and cultured for six days. At the end of the six day culture period, a ⁵¹Cr release cytotoxicity assay is performed as described in Example 1. Effector cells are in vitro stimulated PBMCs and target cells are either virus infected or mock infected autologous PBMCs. Effector to target cell ratios are 10:1, 20:1, 40:1 and 80:1.

### Conclusion

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method of eliciting an immune response against a bovine virus comprising, combining at least one bovine viral epitope and at least one heat shock protein to form a purified epitope/heat shock protein complex, and administering an immune system stimulating amount of said purified epitope-heat shock protein complex to an animal.

2. The method of claim 1 wherein said bovine viral epitope further comprises a supermotif.

3. The method of claim 1 wherein said bovine viral epitope further comprises an allele specific peptide motif.

4. The method of claim 3 wherein said allele specific peptide motif is selected from the group consisting of BoLA-A11, BoLA-A20, BoLA-HD1, BoLA-HD6 and BoLA-HD7.

5. The method claim 1, wherein said bovine viral epitope is between 5 and 25 amino acids in length.

6. The method of claim 1, wherein said bovine viral epitope is between 5 and 15 amino acids in length.

7. The method of claim 1, wherein said viral epitope is between 8 and 10 amino acids in length.

8. The method of claim 1 wherein said epitope is from a virus selected from the group consisting of bovine viral diarrhea virus, bovine respiratory syncytial virus, parainfluenza virus III, bovine corona virus, and bovine rota virus.

9. The method of claim 1 wherein said heat shock protein is selected from the group consisting of HSP 60, HSP 70 and HSP 90 families.

10. The method of claim 9 wherein said heat shock protein is gp96

11. The method of claim 1 wherein said heat shock protein is a heterologous heat shock protein.

12. The method of claim 1 wherein said heat shock protein is a homologous heat shock protein.

13. The method of claim 1 wherein said epitope/heat shock protein complex is formed in vitro.

14. The method of claim 1 wherein said epitope/heat shock protein complex is formed in vivo.

15. The method of claim 1 wherein said epitope is a recombinant epitope

16. The method of claim 1 wherein said epitope is a synthetic peptide

17. The method of claim 16, wherein said synthetic peptide is synthesized by solid phase chemistry.

18. The method of claim 1 wherein said animal is a ruminant.

19. The method of claim 18 wherein said ruminant is a Bovidae.

20. The method of claim 19 wherein said Bovidae is of the genus Bos.

21. A method for eliciting an immune response to a bovine virus comprising, combining at least one bovine virus allele specific peptide motif containing epitope of at least 8-10 amino acids long and a heat shock protein gp96 to form a purified epitope/heat shock protein complex, and administering an immune system stimulating amount of said purified epitope-heat shock protein complex to a ruminant.

22. A composition comprising, a purified epitope/heat shock protein complex containing at least one bovine virus epitope complexed with at least one heat shock protein, and a pharmaceutically acceptable carrier, diluent or excipient.

23. The composition of claim 22, wherein said bovine viral epitope further comprises a supermotif.

24. The composition of claim 22, wherein said bovine viral epitope further comprises an allele specific peptide motif.

25. The composition of claim 24, wherein said allele specific peptide motif is selected from the group consisting of BoLA-A11, BoLA-A20, BoLA-HD1, BoLA-HD6 and BoLA-HD7.

26. The composition claim 22, wherein the bovine viral epitope is between 5 and 25 amino acids in length.

27. The composition of claim 22, wherein the bovine viral epitope is between 5 and 15 amino acids in length.

28. The composition of claim 22, wherein the bovine viral epitope is between 8 and 10 amino acids in length.

29. The composition of claim 22 wherein said epitope is from a virus selected from the group consisting of bovine viral diarrhea virus, bovine respiratory syncytial virus, parainfluenza virus III, bovine corona virus, and bovine rota virus.

30. The composition of claim 22, wherein said heat shock protein is selected from the group consisting of HSP 60, HSP 70 and HSP 90 families.

31. The composition of claim 30 wherein said heat shock protein is gp96

32. The composition of claim 22, wherein said heat shock protein is a heterologous heat shock protein.

33. The composition of claim 22, wherein said heat shock protein is a homologous heat shock protein.

34. The composition of claim 22, wherein said epitope/heat shock protein complex is formed in vitro.

35. The composition of claim 22 wherein said epitope/heat shock protein complex is formed in vivo.

36. The composition of claim 22 wherein said epitope is a recombinant epitope

37. The composition of claim 22 wherein said epitope is a synthetic peptide.

38. The composition of claim 37 wherein the synthetic peptide is synthesized by solid phase chemistry.

39. A composition comprising, a purified epitope/heat shock protein complex containing:
a gp96 heat shock protein;
at least bovine viral epitope of 8-10 amino acids long, said epitope being from a virus selected from the group consisting of bovine viral diarrhea virus, bovine respiratory syncytial virus, parainfluenza virus III, bovine corona virus, and bovine rota virus; and
a pharmaceutically acceptable carrier, diluent or excipeint.
